(19) United States
(12) Patent Application Publication
(10) Pub. No.: US 2020/0093426 A1
(43) Pub. Date: Mar. 26, 2020

(54) METHOD AND SYSTEM OF OBSTRUCTED AREA DETERMINATION FOR SLEEP APNEA SYNDROME

(71) Applicant: National Central University, Taoyuan City (TW)

(72) Inventors: Yi-Chung CHANG, Taoyuan City (TW); Leh-Kiong HUON, Sibu (MY); Van-Truong PHAM, Hai Phong City (VN); Yunn-Jy CHEN, Tapei City (TW); Tiffany Ting-Fang SHIH, Taipei City (TW); Thi-Thao TRAN, Vinh Phuc Province (VN); Chen LIN, Taoyuan City (TW); Men-Tzung LO, Taoyuan City (TW); Pa-Chun WANG, Taipei (TW)

(21) Appl. No.: 16/699,470

(22) Filed: Nov. 29, 2019

Related U.S. Application Data
(63) Continuation-in-part of application No. 14/858,920, filed on Sep. 18, 2015.

(30) Foreign Application Priority Data
May 14, 2015 (TW) 104115403

(51) Int. Cl. A61B 5/00; A61B 5/055

(52) U.S. Cl. CPC A61B 5/4818; A61B 5/055; A61B 2562/0204; A61B 2576/02; A61B 5/7257

(57) ABSTRACT

A method and system of obstructed area determination for sleep apnea syndrome are disclosed. The method comprising obtaining a snoring signal from an individual; obtaining a spectrogram of the snoring signal based on short-time Fourier Transform; obtaining a snoring signal feature based on a harmonic wave of the spectrogram; and obtaining a collapse index by comparing the snoring signal feature against a snoring signal feature-collapse index correlation database. The present invention enables the diagnosis of sleep apnea syndrome and determination the obstructed area for the sleep apnea syndrome with only the snoring signal of an individual during sleep.

### CROSS-REFERENCE TO RELATED APPLICATIONS

This Non-provisional application is a continuation-in-part of U.S. patent application Ser. No. 14/858,920, filed Sep. 18, 2015, which claims the benefit of priority Patent Application No(s). 104115403 filed in Taiwan, Republic of China May 14, 2015.

### FIELD OF THE INVENTION

The invention relates to a method and system of establishment of a correlation database for determining an obstructed area, and more particularly, a method and system of establishment of a correlation database for determining an obstructed area caused by sleep apnea syndrome.

### BACKGROUND OF THE INVENTION

Sleep-disordered breathing is the common sleep problem, which includes primary snoring, upper airway resistance syndrome and obstructive sleep apnea syndrome, and is a risk factor for high blood pressure, heart disease and stroke, and is associated with overall decreased quality of life and wellbeing. The sleep-disordered breathing is most common caused by upper airway obstruction. Snoring, sleep apnea, abnormally low respiratory rate or airway resistance is muscles in the upper airway relax during sleep for a constant volume of inspired air, the air speed through the collapsed region must increase. Whenever there is an increase in air velocity, there is also corresponding to a result of multi-level airway collapse and hard to determine the obstructed area accurately.

The patient underwent examination at snoring and sleep-disordered breathing includes oral examination, for example, Mallampati classification and Friedman classification. Mallampati classification is a simple, noninvasive, inexpensive technique that involves visualization of the oropharynx. It is easy to learn and does not require any special equipment or setting. However, Mallampati classification and Friedman classification may not reliable due to a different “subjectivity”. Muller maneuver and drug-induced sleep endoscopy may provide quantitative information. Drug-induced sleep endoscopy uses progressive doses of anesthesia to pharmacologically induce sleep to the point of the obstruction-causing apnea in a short time frame. In addition, because the patient has to be monitored by an electronic instrument in specific surroundings, the accuracy of the polysomnogram (PSG) data may be reduced because of various physiologic factors of the patient (such as anxiety, tension, or excitement). The polysomnogram test only can provide an apnea hypopnea index of the patient. However, it is not accurate enough to determine whether the patient suffers from the obstructive sleep apnea just based on the apnea-hypopnea index (AHI), and the parameters from the apnea-hypopnea index and lowest blood oxygenation saturation may only relate to some anatomic structures.

TWI413511 describes a method and a computer for aiding determination of obstructive sleep apnea, which may generate a stenosis rate and a flow field pressure distribution of an upper airway of a patient, so as to assist a physician to fast determine whether the patient suffers from the obstructive sleep apnea. TWI442904 describes a method and device also including three fuzzy logic systems so as to analyze the characteristics of sleep apnea, cough and asthma based on these three detected signals. In order to resolve the problems in the traditional obstructive sleep apnea diagnostic method, and people expect a more convenient method to determine sleep apnea syndrome for medical diagnosis. The present invention disclosures a system and method of obstructed area determination for sleep apnea syndrome to provide a better therapy for sleep apnea syndrome based on the obstructed area and the degree of obstruction.

### SUMMARY OF THE INVENTION

The present invention provides a method and system of establishment of a correlation database for determining an obstructed area caused by sleep apnea syndrome. The inventor discovers that snoring is associated with the area of collapse. Therefore, the present invention provides a system and method to establish the snoring signal feature-collapse index correlation database and use the snoring signal feature-collapse index correlation database to determine the area of collapse in obstructive sleep apnea based on the snoring signal corresponding to the obstructed area.

In an embodiment of the invention, the present invention provides a method of establishment of a correlation database for determining an obstructed area caused by sleep apnea syndrome. The method comprises obtaining a plurality of snoring signals and a plurality of dynamic images of respiratory tract from a plurality of snoring events from an individual and recording an obstructed area caused by each of the snoring events based on its corresponding dynamic image of respiratory tract. The method further comprises obtaining a collapse index based on the dynamic image of respiratory tract showing a region of interest where an airway section appears, obtaining a snoring signal feature based on a spectrogram of the snoring signal and generating the correlation database based on the collapse index and the snoring signal feature corresponding to the obstructed area.

In an embodiment of the invention, the present invention provides a system to establish a correlation database for determination of an obstructed area caused by sleep apnea syndrome. The system comprises a dynamic image receiving unit configured to receive a plurality of dynamic images of respiratory tract from a plurality of snoring events from an individual. The system comprises a voice receiving unit configured to receive a plurality of snoring signals in synchronization with the dynamic image of respiratory tract from the snoring events. The system comprises a storage medium configured to store a correlation database. The system comprises a processor unit coupled to the dynamic image receiving unit, the voice receiving unit and storage medium, the processor unit is configured to record an obstructed area caused by each of the snoring events based on the dynamic image of respiratory tract, obtain a collapse index based on the dynamic image of respiratory tract showing a region of interest where an airway section appears, obtain a snoring signal feature based on a spectrogram of the snoring signal and store the collapse index and the snoring signal feature corresponding to the obstructed area in the correlation database.

In an embodiment of the invention, the present invention provides a method of obstructed area determination for sleep apnea syndrome. The method comprises obtaining a snoring signal from an individual and obtaining a spectrogram of the snoring signal by using short-time Fourier transform. The method further comprises obtaining a snoring signal feature based on a harmonic wave of the spectrogram and obtaining a collapse index by comparing the snoring signal feature against a snoring signal feature-collapse index correlation database.

In an embodiment of the invention, the present invention provides a system of obstructed area determination for sleep apnea syndrome. The system comprises a voice receiving unit configured to receive a snoring signal from an individual. The system comprises a storage medium configured to store a snoring signal feature-collapse index correlation database. The system comprises a processor unit coupled to the voice receiving unit and the storage medium, the processor unit is configured to obtain a spectrogram of the snoring signal by using short-time Fourier transform, obtain a snoring signal feature based on a harmonic wave of the spectrogram, and obtain a collapse index by comparing the snoring signal feature against the snoring signal feature-collapse index correlation database.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a flowchart that provides one example of a method of establishment of a correlation database for determining an obstructed area caused by sleep apnea syndrome, according to some embodiments.

FIG. 2 is a block diagram illustrating selected components of a system to establish a correlation database for determination of an obstructed area caused by sleep apnea syndrome, according to some embodiments.

FIG. 3 is a flowchart that provides one example of procedure for processing a dynamic image of respiratory tract performed by the system of FIG. 2, according to some embodiments.

FIG. 4 illustrates an example of a snoring signal segmented by a major constituent analysis, according to various embodiments.

FIG. 5 illustrates one example of a snoring signal for inhalation and expiratory, and a spectrogram of the snoring signal, according to some embodiments.

FIG. 6 illustrates one example of a spectrogram of a group of snoring signals, according to some embodiments.

FIG. 7 illustrates one example of a plurality of dynamic images of respiratory tract and collapse indexes corresponding to the snoring signals of FIG. 6, according to some embodiments.

FIG. 8 illustrates one example of a spectrogram is generated based on a continued breathing sound signal and a curve diagram illustrates a collapse index, according to some embodiments.

FIG. 9 is one example of a spectrogram depicting a correlation between collapse indexes and the snoring signal features for different obstructed areas, according to some embodiments.

FIG. 10 is one example of a statistical chart depicting a correlation between collapse indexes and the snoring signal features for different obstructed areas, according to some embodiments.

FIG. 11 is a flowchart that provides one example of a method of obstructed area determination for sleep apnea syndrome, according to some embodiments.

### DETAILED DESCRIPTION OF THE INVENTION

Having broadly summarized certain features of method and system to establish a correlation database for determination of an obstructed area caused by sleep apnea syndrome of the present disclosure, reference will now be made in detail to the description of the disclosure as illustrated in the drawings. While the disclosure is described in connection with these drawings, there is no intent to limit the disclosure to the embodiment or embodiments disclosed herein. Although the description identifies or describes specifics of one or more embodiments, such specifics are not necessarily part of every embodiment, nor are all various stated advantages associated with a single embodiment. On the contrary, the intent is to cover all alternatives, modifications and equivalents included within the spirit and scope of the disclosure as defined by the appended claims. Further, it should be appreciated in the context of the present disclosure that the claims are not necessarily limited to the particular embodiments set out in the description.

The present invention discloses a method of obstructed area determination for sleep apnea syndrome and a method of establishment of a correlation database for determining the obstructed area. It is understood that the methods provide merely an example of the many different types of functional arrangements that may be employed to implement the operation of the various components of a dynamic image receiving unit or a voice receiving unit, a computer system coupled to the dynamic image receiving unit, the voice receiving unit, and so forth. The execution steps of the present invention may include application specific software which may store in any portion or component of the memory including, for example, random access memory (RAM), read-only memory (ROM), hard drive, solid-state drive, magneto optical (MO), IC chip, USB flash drive, memory card, optical disc such as compact disc (CD) or digital versatile disc (DVD), floppy disk, magnetic tape, or other memory components.

For embodiments, the system comprises a display device, a processing unit, a memory, an input device and a storage medium. The input device is used to provide data such as image, text or control signals to an information processing system such as a computer or other information appliance. In accordance with some embodiments, the storage medium such as, by way of example and without limitation, a hard drive, an optical device or a remote database server coupled to a network, and stores software programs. The memory typically is the process in which information is encoded, stored, and retrieved etc. The processing unit performs data calculations, data comparisons, and data copying. The display device is an output device that visually conveys text, graphics, and video information. Information shown on the display device is called soft copy because the information exists electronically and is displayed for a temporary period of time. The display device includes CRT monitors, LCD monitors and displays, gas plasma monitors, and televisions. In accordance with such embodiments of present invention, the software programs are stored in the memory and executed by the processing unit when the computer system executes an obstructed area determination method for sleep apnea syndrome. Finally, information provided by the processing unit, and presented on the display device or stored in the storage medium.

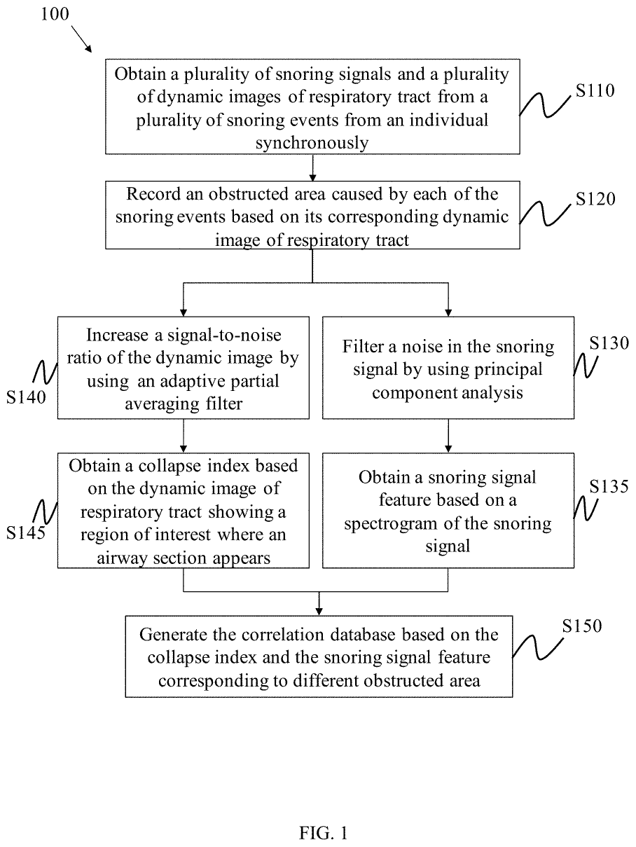

FIG. 1 is a flowchart illustrating a method 100 to establish a correlation database to determine an obstructed area for sleep apnea syndrome. If embodied in software, each block depicted in FIG. 1 represents a module, segment, or portion of code that comprises program instructions stored on a non-transitory computer readable medium to implement the specified logical function(s). In this regard, the program instructions may be embodied in the form of source code that comprises statements written in a programming language or machine code that comprises numerical instructions recognizable by a suitable execution system such as a processor in a computer system or other system such as the one shown in FIG. 1. The machine code may be converted from the source code, etc. If embodied in hardware, each block may represent a circuit or a number of interconnected circuits to implement the specified logical function(s).

Although the flowchart 100 of FIG. 1 shows a specific order of execution, it is understood that the order of execution may differ from that which is depicted. Beginning with step S110, obtaining a plurality of snoring signals via a microphone during a plurality of snoring events and filtering a noise in the snoring signal by using principal component analysis with the processor, then saving the plurality of snoring signals in a storage unit; obtaining a plurality of dynamic images of respiratory tract via a processor from a MRI apparatus during the snoring events and increasing a signal-to-noise ratio of the dynamic image by using an adaptive partial averaging filter with the processor, then saving the plurality of dynamic images in the storage unit; setting a calibrate reference by using the noises caused by the MRI apparatus, at least one time reference to calibrate a time difference between the dynamic image and the snoring signal. In step S120, determining an obstructed area caused by each of the snoring events based on its corresponding dynamic image of respiratory tract with the processor. In step S130, determining a collapse index based on the dynamic image of respiratory tract showing a region of interest having an airway section with the processor; In step S135, a snoring signal feature is obtained based on the snoring signal in a spectrogram. In step S140, determining a snoring signal feature based on a spectrogram of the snoring signal with the processor, the snoring signal feature is a vibration time duration of soft tissue; In step S145, a collapse index is obtained based on the dynamic image of respiratory tract showing a region of interest where an airway section appears. In step S150, generating a correlation database based on the collapse index and the snoring signal feature corresponding to the obstructed area with the processor; and obtaining an obstructed area caused by sleep apnea syndrome patient's the collapse index and the snoring signal feature corresponding to the obstructed area, and compare to the correlation database to determine the correlation between the collapse index and the vibration time duration of soft tissue, then according to the correlation value to determine the sleep apnea syndrome patient's the site of airway collapse.

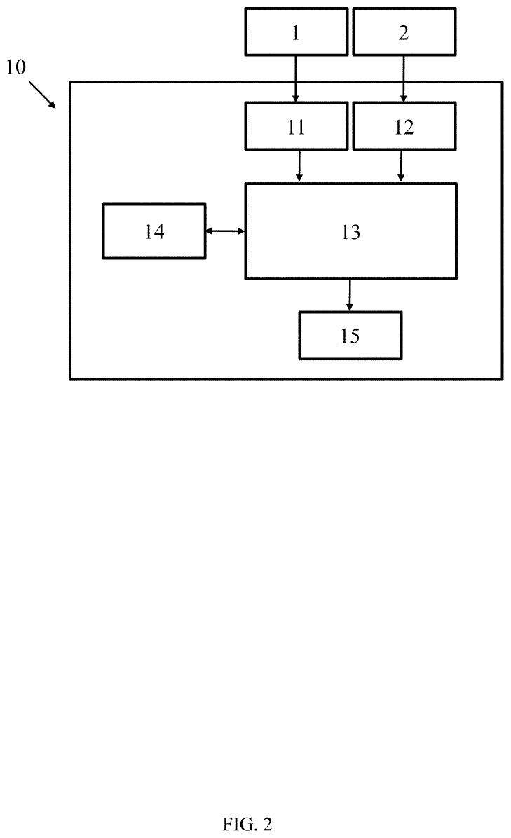

FIG. 2 is a block diagram illustrating selected components of a system 10 to establish a correlation database for determination of an obstructed area caused by sleep apnea syndrome, and the interaction between these components. Reference is made to FIG. 1, which is a flowchart 100 in accordance with one embodiment to establish a correlation database. It is understood that the flowchart of FIG. 1 provides merely an example of the many different types of functional arrangements that may be employed to implement the operation of the system 10 (FIG. 2) as described herein. The system 10 to establish the correlation database for determination of an obstructed area caused by sleep apnea syndrome comprises a dynamic image receiving unit 11, a voice receiving unit 12, a processor unit 13, a storage medium 14 and an output device 15.

With reference to FIGS. 1 and 2, the system 10 comprises the dynamic image receiving unit 11 is electrically coupled to the dynamic image capture device 1. The voice receiving unit 12 is electrically coupled to the voice capture device 2. The dynamic image capture device 1, for example, Magnetic Resonance Imaging (MRI) is a medical imaging procedure dynamic images that uses strong magnetic fields and radio waves to produce images of organs and internal structures in the body. The voice capture device 2, for example, is a microphone device.

Please refer FIG. 1, a plurality of snoring events occur while an individual is sleeping. In step S110, the dynamic image capture device 1 captures a plurality of dynamic images of respiratory tract (e.g., upper respiratory tract) and the voice capture device 2 captures a plurality of snoring signals synchronously while each individual is sleeping, wherein the plurality of dynamic images of respiratory tract and the plurality of snoring signals are from the plurality of snoring events. The dynamic image receiving unit 11 receives the plurality of dynamic images corresponding from the plurality of snoring events from each individual. The voice receiving unit 12 receives the plurality of snoring signals in synchronization with the plurality of dynamic images of respiratory tract from the plurality of snoring events. The dynamic image receiving unit 11 sends the dynamic images of respiratory tract to the processor unit 13 and the voice receiving unit 12 sends the snoring signals to the processor unit 13. In some embodiments, the dynamic image receiving unit 11 and the voice receiving unit 12 obtains the plurality dynamic images or voice signal as a baseline value before the individual falls asleep for further processing.

The processor unit 13 is electrically coupled to the dynamic image receiving unit 11, the voice receiving unit 12 and the storage medium 14. The storage medium 14 is configured to store a correlation database, and the correlation database can be a snoring signal feature-collapse index correlation database.

In step S120, the processor unit 13 records an obstructed area caused by each of snoring events based on its corresponding dynamic image of respiratory tract in the correlation database. The obstructed area is determined by polysomnography, clinical judgment or other clinical examination. The obstructed area may include, for example, retropalatal, retroglossal, and combined (retropalatal with retroglossal). The definition of the retropalatal is from inferior margin of hard palate to inferior margin of uvula. The definition of the retroglossal is from inferior margin of uvula to upper margin of epiglottis. Specifically, conventional diagnoses for sleep apnea syndrome only determine whether it is central apnea or obstructive apnea, or the degree of the syndrome (i.e., apnea-hypopnea index, AHI). In previous study neither do they attempt to determine the area of obstruction where the syndrome occurs. The main cause for obstructive sleep apnea is airway obstruction. However, the obstruction may occur in any places of a patient's airway, and sometimes, there may be multiple obstructions appearing in different places.

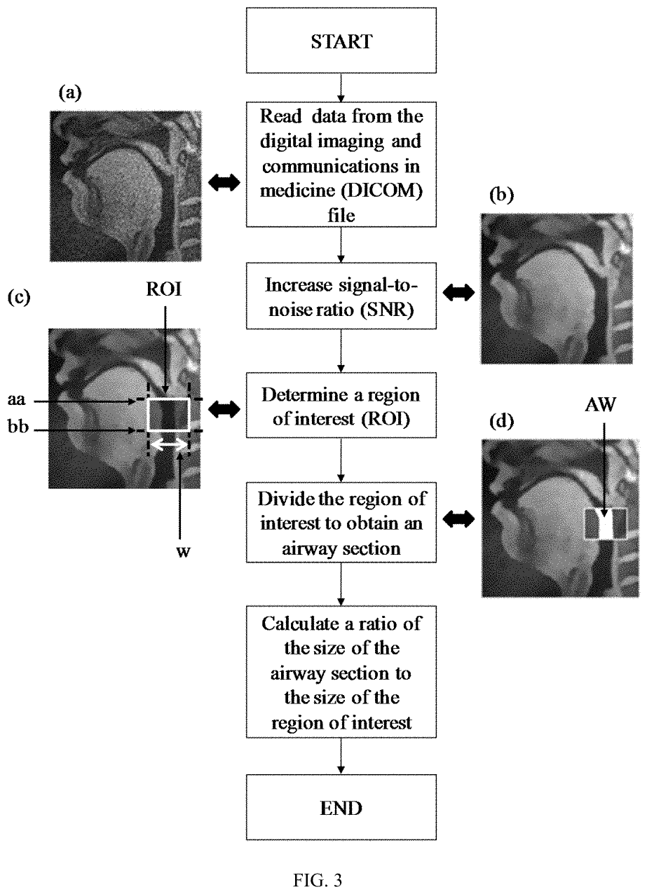

Attention is directed to FIG. 3 is a flowchart that provides one example of procedure for processing a dynamic image of respiratory tract performed by the system of FIG. 2, according to various embodiments. As seen in FIG. 3, the dynamic image of respiratory tract is such as, for example, the images of respiratory tract in sagittal, coronal, upper horizontal, middle horizontal, or lower horizontal view. In some embodiment, the dynamic image of respiratory tract is the sagittal image. Magnetic Resonance Imaging (MRI) is an established technique for both spectroscopy and imaging such as, for example, the dynamic image. Image quality and time efficiency of data collection is inversely related in the fast low-angle shot (FLASH) imaging method. The dynamic images were obtained by using the fast low-angle shot imaging method which allowed for a drastic shortening of the measuring times with a substantial loss in image quality.

Reference is made to FIG. 3(a), FIG. 3(a) illustrates an original image, wherein the original image is read from the digital imaging and communications in medicine (DICOM) file. The processor unit 13 increases signal-to-noise ratio (SNR) for the dynamic images of respiratory tract. The processor unit 13 further processes the dynamic images of respiratory tract by using an adaptive partial averaging filter (APAF) (step S140). For the adaptive partial averaging filter, please read “Novel noise reduction filter for improving visibility of early computed tomography signs of hyperacute stroke: evaluation of the filter's performance-preliminary clinical experience,” Radiation Medicine, vol. 25, pp. 247-254, 2007 for more details. FIG. 3(b) illustrates a denoise image processed by the adaptive partial averaging filter.

In step S145, the processor unit 13 obtains a collapse index based on the dynamic image of respiratory tract showing a region of interest (ROI) where an airway (AW) section appears. FIG. 3(c) illustrates a region of interest where an airway section appears, according to some embodiments. For example, the region of interest is defined by the upper extremity aa as inferior margin of uvula and the lower extremity bb as upper margin of epiglottis. In addition, a center is the section in the central part of airway section between the upper extremity aa and the lower extremity bb. The width (W) is the triple of an average width of the airway section of the upper extremity aa and the lower extremity bb. The width may include by way of example and without limitation, determined by the average width of respiratory tract in dynamic image while the patient is awake. However, it should be understood that the invention is not limited to the specific details of this example.

The dynamic image is increased the signal-to-noise ratio by image denoising process to improve the accuracy of image segmentation. FIG. 3(d) illustrates a result of image segmentation, according to some embodiments. The processor unit 13 segments the region of interest to obtain the airway section via utilizing active contour model (ACM) approach. Active contour is known to be one of the most powerful methods in image segmentation, and has been extensively used in a wide range of applications including computer vision, pattern recognition and medical imaging. Especially, in medical imaging, active contour model is commonly utilized to segment and track the desired organs. In the invented system, the active contour model is used to delineate the boundaries of airway section. To this end, an initial contour is first defined inside the ROI by guidance from the system or interaction with user. Then, the image information i.e., image intensity, is utilized to guide the evolution of the contour, and the airway section is extracted. For active contour model, please refer to the well-known article “Active Contours without Edges,” IEEE Transactions on Image Processing, vol. 10, No. 2, pp. 266-277, February 2001.

In some embodiments, the collapse index is a ratio of the size of the airway section to the size of the region of interest. As mentioned above, the region of interest is defined then the airway section is segmented from the region of interest. Finally, the collapse index is calculated based on the size of the region of interest and the size of the airway section. However, it should be understood that the invention is not limited to the specific details of this example.

In step S135, the processor unit 13 obtains the snoring signal features based on the spectrogram of each snoring signal. Since the snoring signals and the dynamic images of respiratory tract are captured synchronously. Noise will be inevitably introduced in the image acquisition process. In MRI, a major form of noise is hiss caused by random electrons that, heavily influenced by heat, stray from their designated path. These stray electrons influence the voltage of the output signal and thus create detectable noise and denoising is an essential step to improve the image quality. The processor unit 13 further filters a noise in the snoring signal by using principal component analysis (PCA) (step S130).

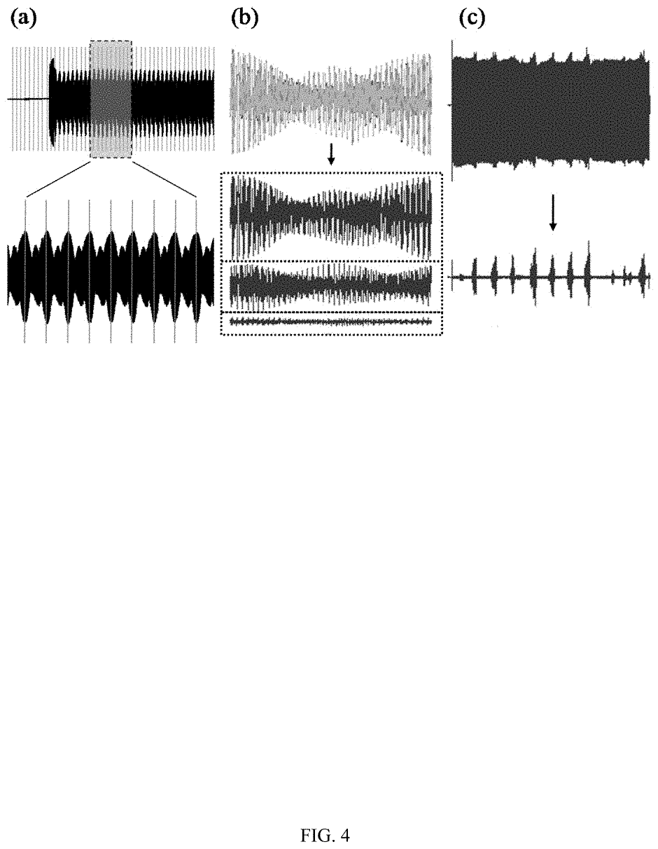

Reference is made to FIG. 4, FIG. 4(a) shows the snoring signal and a drawing of partial enlargement, wherein the duration for capturing the snoring signal is five seconds. The dynamic image capture device 1 captures the snoring signals when the images are scanned from different slices to generate a data matrix before processing a major constituent analysis. FIG. 4(b), which shows three different constituent parts, wherein the data matrix is divided into three different constituent parts by processing the major constituent analysis. The apparatus noise is selected according to the characteristic noises of the apparatus when the apparatus under operating condition. FIG. 4(c) illustrates a spectrogram of an original snoring signal and a snoring signal after denoising, according to some embodiments. The length of the snoring signal after denoising is about fifty two seconds. One of the apparatus noises is result from the image acquisition process. The noise could act as a time reference for calibrating the dynamic images and the snoring signals. The time difference may be incurred because of a gap between image recording process and voice recording process. As illustrated in FIG. 4(a), the vertical lines in grey are the time references for the snoring signals and dynamic images. It is used to calibrate a time difference between recorded dynamic images and recorded snoring signals. The processor unit 13 further adjusts the time difference between the recorded dynamic images and the recorded snoring signals based on a plurality of time references. Furthermore, in FIG. 4, when the microphone device records the snoring sound, the noise caused by the MRI apparatus operated will also be recorded. The noise is majorly contributed by the vibration of the gradient coil, it is periodic. Given the nature, as depicted by the vertical grey line in FIG. 4(a), the beginning and the end points of each segment can serve as a time reference to calibrate a time difference between recorded dynamic images and recorded snoring signals.

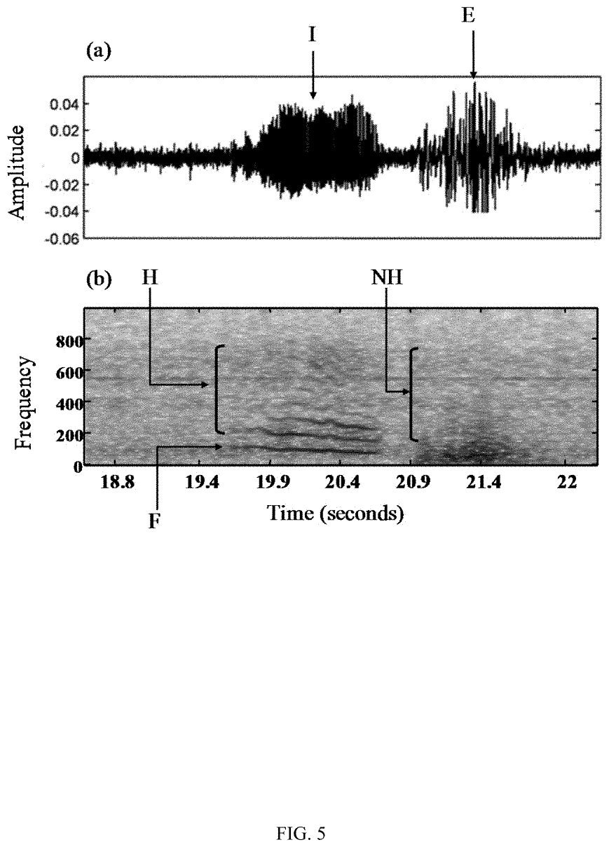

As seen in FIG. 5 illustrates one example of a snoring signal after denoising, according to some embodiments. In FIG. 5(a) illustrates different types of wave motion with two regions which comprises inhalation (I) and expiratory (E). FIG. 5(b) illustrates one example of the spectrogram of the snoring signal, which is generated by using short-time Fourier transform and Gaussian sliding window, according to some embodiments. The length of Gaussian sliding window is 0.1 second. A length between two consecutive windows is 0.005 seconds. As seen in FIG. 5(b), H region is harmonic wave; F region is a basis wave; NH region is non harmonic wave.

A study by the inventor, when the patient is asleep, the muscle in the upper airway become less active and tension is lost. The harmonic wave (H region) is generated with snoring sounds by vibrating the soft tissues of the upper airway, typically during inspiratory breath. However, the harmonic wave is not generated during expiratory breath (NH region). Therefore, the time of inspiratory breath and the time of expiratory breath, and a time period of snoring is determined by the spectrogram of the snoring signal. A time period of the harmonic wave is corresponding to the vibration time duration of soft tissue as a snoring signal feature.

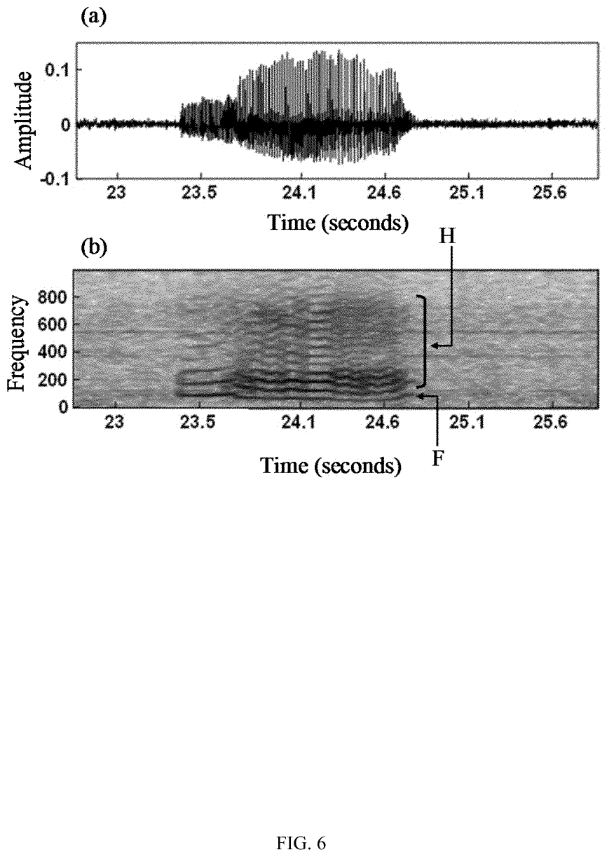

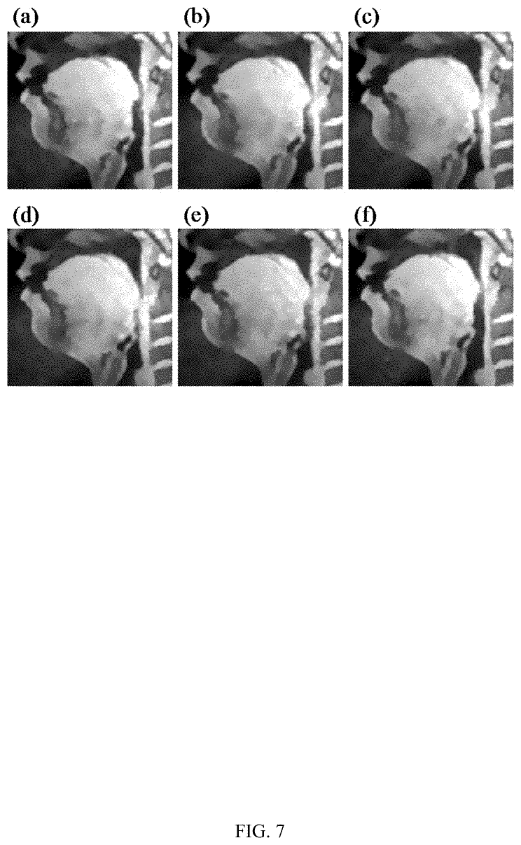

Please refer FIG. 6 and FIG. 7, in FIG. 6(a) illustrates one example of a group of snoring signals, according to some embodiments. FIG. 6(b) illustrates a spectrogram based on the group of snoring signals in FIG. 6(a), according to some embodiments. Reference is made to FIG. 6(a) and FIG. 6(b), which shows one example of the harmonic wave. The time period of the harmonic wave is corresponding to the vibration time duration of soft tissue with snoring sounds. In the same way, H region is harmonic wave and F region is a basis wave. Reference is made to FIG. 7(a) to FIG. 7 (f), illustrates one example of a plurality of dynamic images of respiratory tract and collapse indexes corresponding to the snoring signals of FIG. 6, according to some embodiments. As seen in FIG. 7(a), at 23^{rd} second, the collapse index is 15.8%. As seen in FIG. 7(b), at 23.5^{th} second, the collapse index is 9.8%. As seen in FIG. 7(c), at 24.1^{st} second, the collapse index is 6.8%. As seen in FIG. 7(d), at 24.6^{th} second, the collapse index is 5.4%. As seen in FIG. 7(e), at 25.1^{st} second, the collapse index is 10.3%. As seen in FIG. 7(f), at 25.6^{th} seconds, the collapse index is 16.4%. As illustrated in FIG. 7(a) to FIG. 7(c), the airway continues to collapse and the collapse index decrease. With further reference to FIG. 6, the time of begging snoring is determined. FIG. 7(d) illustrates one example showing the collapse index is decreasing to its smallest point and eventually the snoring sound stops at the later stage of inspiratory. FIG. 7(d) to FIG. 7(f) illustrates stopping the snoring sound when a collapsed airway gradually expands again, and the harmonic wave is not generated in the spectrogram.

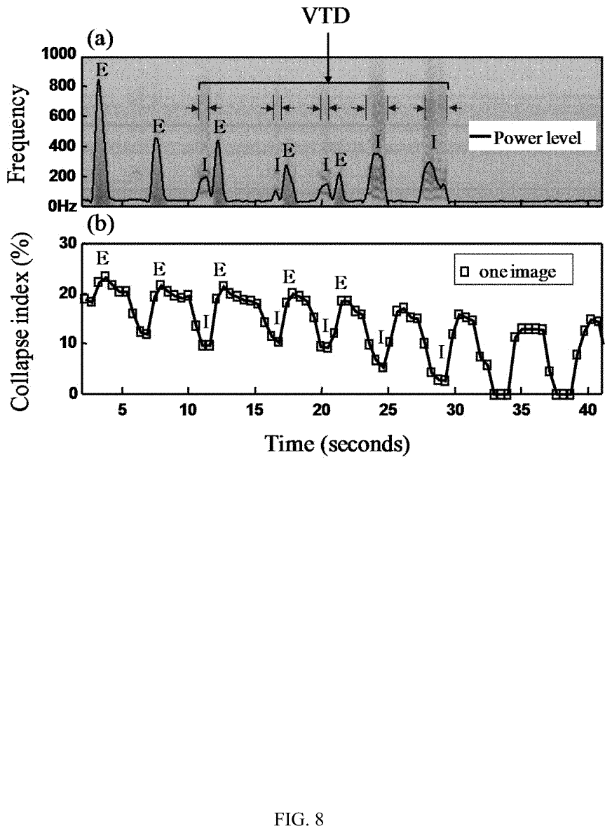

As seen in FIG. 8(a), a spectrogram is generated based on a continued breathing sound signal. Two regions are inhalation (I) and expiratory (E) identified in the spectrogram. Therefore, the snoring signal is identified based on the harmonic wave whether generated, and five vibration time durations (VTD) of soft tissue are defined. A time period of the harmonic wave is corresponding to the vibration time duration of soft tissue as a snoring signal feature. As seen in FIG. 8(b), a curve diagram illustrates a collapse index based on the dynamic images of respiratory tract and continued breathing sound signals, wherein a frequency sampling is 0.5 Hz. By observing two snoring events in the curve diagram, the airway gradually narrows (the collapse index is decreasing) at around 25^{th} second and 30^{th} second according to five vibration time durations of soft tissue and corresponding dynamic images of respiratory tract and collapse indexes. The obstructed area can be observed as a combination of retropalatal and retroglossal obstruction when the collapse index is lower than 10% and the vibration time duration of soft tissue is longer. Furthermore, the retroglossal area that is totally obstructed at around 30^{th} to 40^{th} second and stopping snoring.

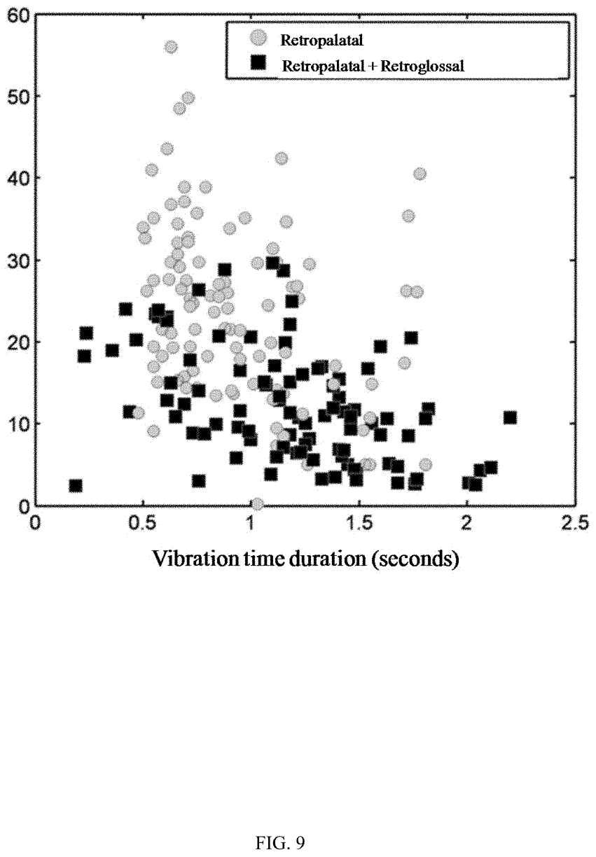

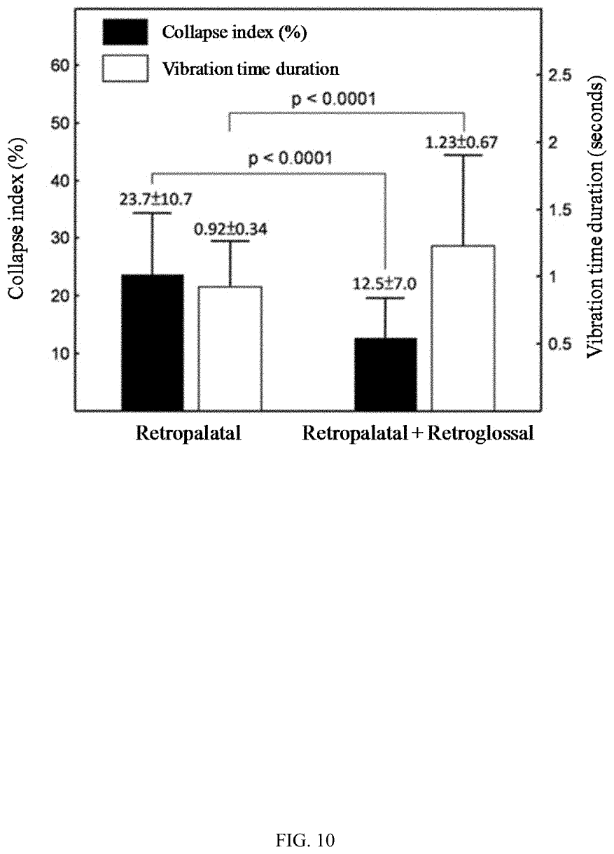

As seen in FIGS. 9 and 10, the processor unit 13 (FIG. 2) stores the collapse indexes and the snoring signal features corresponding to different obstructed areas in the correlation database 14 (step S150). As seen in FIG. 9, the correlation database 14 (FIG. 2) provides different obstructed areas corresponding to different collapse indexes and the vibration time duration of soft tissue. An obstructed area is considered to a collapse index and a vibration time duration of soft tissue. As seen in FIG. 10, a statistical chart depicts different collapse indexes and the vibration time duration of soft tissue representing different obstructed areas. Moreover, there is the correlation between the collapse index and the vibration time duration of soft tissue. The statistical chart depicts different collapse indexes and snoring signal features representing different obstructed areas, for example, the collapse index of retropalatal obstruction is about 24%±11%; the collapse index of combined retropalatal and retroglossal obstruction is about 13%±7% [P≤0.0001]. Therefore, it proves that the correlation database 14 (FIG. 2) determines the area of airway collapse in obstructive sleep apnea according to the correlation between the collapse index and the vibration time duration of soft tissue.

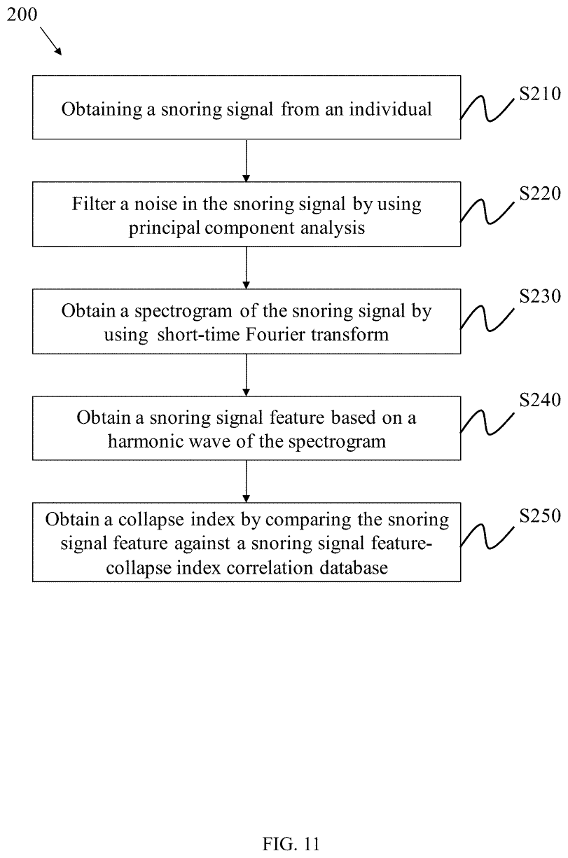

FIG. 11 is a flowchart describing one example of a method of obstructed area determination for sleep apnea syndrome performed by a system of obstructed area determination for sleep apnea syndrome, according to some embodiments. The method 200 determines the area of airway collapse in obstructive sleep apnea based on the correlation database obtained by the method and system as mentioned previously. The system of obstructed area determination for sleep apnea syndrome maybe same as the system to establish a correlation database for determination of an obstructed area caused by sleep apnea syndrome 10, but also can be an independent system. The system of obstructed area determination for sleep apnea syndrome comprises a voice receiving unit, a storage medium, a processor unit and an output device. However, a dynamic image receiving unit is not necessarily for the system of obstructed area determination for sleep apnea syndrome.

In step S210, a voice capture device captures a snoring signal from an individual. Then the voice receiving unit is coupled to the voice capture device to receive the snoring signal from the individual and sends the snoring signal to the processor unit. A storage medium is configured to store a snoring signal feature-collapse index correlation database. The snoring signal feature-collapse index correlation database is established based on the method and system to establish the correlation database for determining the obstructed area caused by sleep apnea syndrome as mentioned previously.

The processor unit is electrically coupled to the voice receiving unit, the storage medium and the output device. The processor unit transforms the snoring signal into a spectrogram after the processor unit receives the snoring signal from the individual (step S230), wherein the spectrogram is generated by using short-time Fourier transform with Gaussian sliding window. As mention previously, the processor unit further selectivity filters noise in the snoring signal by using principal component analysis (PCA) (step S220).

Then, the processor unit obtains a snoring signal feature based on a harmonic wave of the spectrogram (step S240), wherein the snoring signal feature is a vibration period duration of soft tissue. Finally, the processor unit obtains a collapse index by comparing the snoring signal feature against the snoring signal feature-collapse index correlation database (step S250). The processor unit determines the obstructed area for sleep apnea syndrome based on the collapse index and outputs to the output devise. Note that the output device may refer to any device that is capable of both displaying video content and outputting audio content (e.g., a flat panel display with integrated speakers).

Obstructive sleep apnea (OSA) is the most common type of sleep apnea and is caused by obstruction of the upper airway. Obstructive sleep apnea occurs when the muscles that support the soft tissues in the throat, such as the tongue and soft palate, temporarily relax. When these muscles relax, the airway is narrowed or closed, and breathing is momentarily cut off. The inventor discovers that snoring is associated with the area of collapse. Therefore, the present invention provides a system and method to establish the snoring signal feature-collapse index correlation database for determining the area of collapse in obstructive sleep apnea with the snoring signal or the dynamic image monitoring. The present invention disclosures an effective method for identifying the patient whether may have obstructive sleep apnea and determining the area of collapse. Clinically, there are more than 20 treatments for sleep apnea syndrome. If the areas where the obstructions occur can be ascertained, doctors will be able to make a precise treatment plan targeting the patient's syndrome. Consequently, the medical resource will be less wasted and also the object of precision medicine can be achieved.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A method for determining an obstructed area caused by sleep apnea syndrome, comprising
obtaining a plurality of snoring signals via a microphone during a plurality of snoring events and filtering a noise in the snoring signal by using principal component analysis with the processor, then saving the plurality of snoring signals in a storage unit;
obtaining a plurality of dynamic images of respiratory tract via a processor from a MRI apparatus during the snoring events and increasing a signal-to-noise ratio of the dynamic image by using an adaptive partial averaging filter with the processor, then saving the plurality of dynamic images in the storage unit;
setting a calibrate reference by using the noises caused by the MRI apparatus, at least one time reference to calibrate a time difference between the dynamic image and the snoring signal;
determining an obstructed area caused by each of the snoring events based on its corresponding dynamic image of respiratory tract with the processor;
determining a collapse index based on the dynamic image of respiratory tract showing a region of interest having an airway section with the processor;
determining a snoring signal feature based on a spectrogram of the snoring signal with the processor, the snoring signal feature is a vibration time duration of soft tissue;
generating a correlation database based on the collapse index and the snoring signal feature corresponding to the obstructed area with the processor; and
obtaining an obstructed area caused by sleep apnea syndrome patient's the collapse index and the snoring signal feature corresponding to the obstructed area, and compare to the correlation database to determine the correlation between the collapse index and the vibration time duration of soft tissue, then according to the correlation value to determine the sleep apnea syndrome patient's the site of airway collapse.

2. The method of claim 1, the site of airway collapse include the retropalatal or the combined retropalatal and retroglossal obstruction.

3. The method of claim 1, wherein the dynamic image is a sagittal image.

4. The method of claim 1, wherein the airway section is segmented from the region of interest by using active contour model.

5. The method of claim 1, wherein the collapse index is a ratio of the size of the airway section to the size of the region of interest.

6. The method of claim 1, wherein the spectrogram of the snoring signal is generated by using short-time Fourier transform and Gaussian sliding window.

7. The method of claim 1, wherein the snoring signal feature is obtained based on a harmonic wave of the spectrogram.

8. A system to establish a correlation database for determination of an obstructed area caused by sleep apnea syndrome, comprising:
a processor configured to receive a plurality of dynamic images of respiratory tract from a MRI apparatus during a plurality of snoring events;
a microphone configured to receive a plurality of snoring signals during the snoring events; and
a storage medium, configured to store a correlation database;
wherein the processor is coupled to the microphone and storage medium, configured to obtain at least one time reference from noises caused by the MRI apparatus to calibrate a time difference between the dynamic image and the snoring signal, determine an obstructed area caused by each of the snoring events based on the dynamic image of respiratory tract, obtain a collapse index based on the dynamic image of respiratory tract showing a region of interest having an airway section, obtain a snoring signal feature based on a spectrogram of the snoring signal and store the collapse index and the snoring signal feature corresponding to the obstructed area in the correlation database, and
wherein the correlation database is used to determine an area of collapse in obstructive sleep apnea based on the snoring signal feature corresponding to the obstructed area.

9. The system of claim 8, wherein the collapse index is a ratio of the size of the airway section to the size of the region of interest.

10. The system of claim 8, wherein the snoring signal feature is a vibration time duration of soft tissue.
